(19) United States
(12) Patent Application Publication
(10) Pub. No.: US 2023/0076349 A1
(43) Pub. Date: Mar. 9, 2023

(54) Method, Apparatus And Recording Medium For Controlling Laboratory Test

(71) Applicant: THE CATHOLIC UNIVERSITY OF KOREA INDUSTRY-ACADAMIC COOPERATION FOUNDATION, Seoul (KR)

(72) Inventors: Young Jin KIM, Busan (KR); Min Woo PARK, Suwon-si (KR)

(21) Appl. No.: 17/893,683

(22) Filed: Aug. 23, 2022

(30) Foreign Application Priority Data
Aug. 24, 2021 (KR) 10-2021-0111941

(51) Int. Cl. G16H 10/40; G16H 50/20; G06N 20/20

(52) U.S. Cl. CPC G16H 10/40; G16H 50/20; G06N 20/20

(57) ABSTRACT

The disclosure relates to a test control device, method, and recording medium and may particularly provide a test control device, method, and recording medium that monitors a test progress process based on the processing time (also referred to as a turnaround time (TAT)) of an emergency room sample or per-progress step processing time information about each test performed in a laboratory. There is also provided a test control device, method, and recording medium for predicting the test end time of each specimen using a prediction model.

### CROSS-REFERENCE TO RELATED APPLICATION

This application claims priority from Korean Patent Application No. 10-2021-0111941, filed on Aug. 24, 2021, which is hereby incorporated by reference for all purposes as if fully set forth herein.

### BACKGROUND

### Field

The present embodiments provide a test control device, method, and recording medium.

### Description of Related Art

Recently, with the development of advanced equipment, the importance of diagnostic medicine increases. In particular, in diagnostic tests that have relied on manual labor, such as blood tests and urine tests, a highly automated laboratory system is required to handle rapidly increasing samples with a limited budget and to provide high-quality test results. Further, as the recent rapid development of health information and communication technology (ICT) and the acceleration of convergence with medical services and systems, the concept of the hospital information system as a medical infrastructure is expanding and, as the demand for quality enhancement of medical service and optimized hospital operation increases, there are ongoing efforts to recognize informatization as a major infrastructure for hospital operation and to effectively manage it.

Most outpatient treatment consists of reception, (test), treatment, test, and payment. In the case of the first visit, treatment is impossible without a blood test on the same day so that during outpatient treatment, the nurse in charge predicts the end point of the test result heuristically and adjusts the order of treatment appropriately. However, due to the limitations of the current hospital information system, nurses have a lot of difficulties in coordinating the order of treatment according to the prediction and delivering it objectively. Thus, patients have no other choice but to wait at the treatment room. Further, thousands of samples are being produced and processed per hour in the hospital laboratory. Although the progress time is recorded in the hospital information system, since they are too many and are provided in the form of a list, it is difficult for a person to find out whether there is an abnormality one by one, management is tricky, and various accidents may occur accordingly.

Therefore, a need exists for a test control technique for monitoring the status of tests being performed in the laboratory and predicting the test end time to support medical treatment services in the hospital and efficiently operate examination equipment in the laboratory.

### BRIEF SUMMARY

In the foregoing background, the present embodiments aim to provide a test control device, method, and recording medium for monitoring the status of tests being performed in the laboratory and predicting the test end time.

To achieve the foregoing objectives, in an aspect, the present embodiment may provide a test control device comprising a receiving unit receiving operation information from a hospital information system (HIS) and a total laboratory automation system (TLA), a monitoring unit calculating per-progress step processing time information for each test performed in a laboratory based on the received operation information and monitoring a test progress process of the laboratory based on the processing time information, a predicting unit predicting a test end time of each specimen from device state information and specimen sample information collected in real-time using a prediction model trained with the operation information updated at a predetermined period, and a generating unit generating output information for visualizing and displaying status information about the laboratory.

In another aspect, the present embodiment may provide a test control method comprising a receiving step receiving operation information from a hospital information system (HIS) and a total laboratory automation system (TLA), a monitoring step calculating per-progress step processing time information for each test performed in a laboratory based on the received operation information and monitoring a test progress process of the laboratory based on the processing time information, a predicting step predicting a test end time of each specimen from device state information and specimen sample information collected in real-time using a prediction model trained with the operation information updated at a predetermined period, and a generating step generating output information for visualizing and displaying status information about the laboratory.

In another aspect, the present embodiment may provide a recording medium storing a program for executing a test control method, comprising a monitoring function calculating per-progress step processing time information for each test performed in a laboratory based on operation information received from a hospital information system (HIS) and a total laboratory automation system (TLA) and monitoring a test progress process of the laboratory based on the processing time information, a predicting function predicting a test end time of each specimen from specimen sample information and device state information collected in real-time, using a prediction model trained with the operation information updated at a predetermined period, and a generating function generating output information for visualizing and displaying status information about the laboratory.

According to the present embodiments, there may be provided a test control device, method, and recording medium for monitoring the status of tests being performed in the laboratory and predicting the test end time.

### DESCRIPTION OF DRAWINGS

The above and other aspects, features, and advantages of the disclosure will be more clearly understood from the following detailed description, taken in conjunction with the accompanying drawings, in which:

FIG. 1 is a view exemplarily illustrating a system to which a test control device is applicable according to an embodiment of the disclosure;

FIG. 2 is a view illustrating a configuration of a test control device according to an embodiment of the disclosure;

FIG. 3 is a flowchart illustrating an overall operation of a test control method according to an embodiment of the disclosure;

FIG. 4 is a view illustrating an example of an emergency process processing operation of a test control device according to an embodiment of the disclosure;

FIG. 5 is a flowchart illustrating an algorithm used to calculate a time of processing an emergency room sample by a test control device according to an embodiment of the disclosure;

FIG. 6 is a view illustrating an example of a main screen for describing output information of a test control device according to an embodiment of the disclosure;

FIG. 7 is a view illustrating an example of a biochemical screen for describing output information of a test control device according to an embodiment of the disclosure;

FIG. 8 is a view illustrating an example of an immunology screen for describing output information of a test control device according to an embodiment of the disclosure;

FIG. 9 is a view illustrating an example of a test status screen of predicting and using a test end time of a test control device according to an embodiment of the disclosure;

FIG. 10 is a flowchart illustrating a test control method according to an embodiment of the disclosure; and

FIG. 11 is a view conceptually illustrating a configuration of a recording medium according to an embodiment of the disclosure.

### DETAILED DESCRIPTION

The disclosure relates to a test control device, method, and recording medium.

Hereinafter, embodiments of the disclosure are described in detail with reference to the accompanying drawings. The same or substantially the same reference denotations are used to refer to the same or substantially the same elements throughout the specification and the drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the disclosure. These denotations are provided merely to distinguish a component from another, and the essence of the components is not limited by the denotations in light of order or sequence.

In describing the positional relationship between components, when two or more components are described as “connected”, “coupled” or “linked”, the two or more components may be directly “connected”, “coupled” or “linked””, or another component may intervene. Here, the other component may be included in one or more of the two or more components that are “connected”, “coupled” or “linked” to each other.

In relation to components, operational methods or manufacturing methods, when A is referred to as being “after,” “subsequent to,” “next,” and “before,” A and B may be discontinuous from each other unless mentioned with the term “immediately” or “directly.”

When a component is designated with a value or its corresponding information (e.g., level), the value or the corresponding information may be interpreted as including a tolerance that may arise due to various factors (e.g., process factors, internal or external impacts, or noise).

In the disclosure, the hospital information system (HIS) may refer to an information system or a set of information systems that support medical activities. The hospital information system may be a computer system that supports tasks directly related to medical care centered on an electronic medical record (EMR) in the hospital and may further include support functions, such as a workflow system, that enable a flexible hospital workflow. As such, the hospital information system may refer to a system that computerizes the hospital and automates all tasks.

In the disclosure, the total laboratory automation system (TLA) may refer to a system that automates the entire process from test request to blood collection, transport, test, and analysis result reporting. Specifically, in the total laboratory automation system, all the steps from the test including test blood collection, centrifugation, closure opening, and online dispensing to result reporting are automatically connected. In other words, the total laboratory automation system configures test equipment, which are conventionally operated independently, organically in conjunction with one automated line and provide the best service by minimized test time and speedy information transfer.

In the disclosure, the test control device may refer to a server that collects and stores various types of information for operation from the hospital information system (HIS) and the total laboratory automation system (TLA) and may mean a HAS server. In the disclosure, the turnaround time (TAT) of the emergency room sample is a kind of index managed by the hospital laboratory and may mean a time to be observed from a predetermined time point to the end of the test. Specifically, in the case of St. Vincent's Hospital, the emergency room TAT is set to 1 hour from the arrival (provisional reception) of the emergency room sample at the laboratory to the final test completion.

In the disclosure, the laboratory sample processing process may mean being performed in the order of blood collection-provisional reception (arrival of the sample at the laboratory)-reception (insertion of the sample into test equipment)-final reporting.

Hereinafter, embodiments of the disclosure are described in detail with reference to the accompanying drawings.

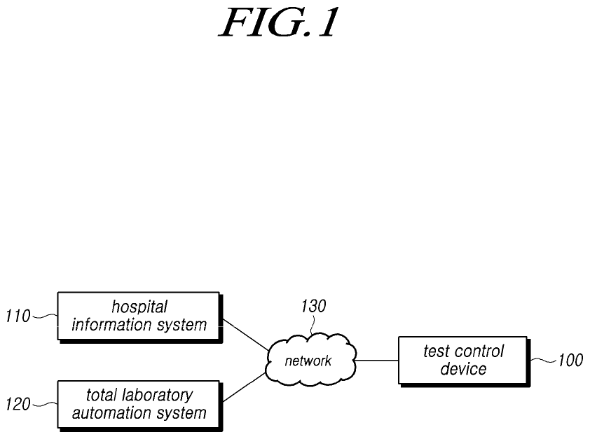

FIG. 1 is a view exemplarily illustrating a system to which a test control device is applicable according to an embodiment of the disclosure.

Referring to FIG. 1, according to an embodiment of the disclosure, a system to which the test control device 100 may be applied may include a hospital information system 110, a total laboratory automation system 120, and a network 130.

According to an embodiment of the disclosure, the test control device 100 hardwarewise has the same configuration as a conventional web server or web application server or WAP server. However, the test control device 100 may softwarewise be implemented through any language, such as C, C++, Java, PHP, .Net, Python, Ruby, and may include program modules that perform various functions.

Further, the test control device 100 may be implemented by way of a server program that is provided in various ways according to operating systems, such as DOS, Windows, Linux, UNIX, and Macintosh on general server hardware and, as a representative examples, may use a website used in the Windows environment or Internet information server (IIS), and Apache, Nginx, or Light HTTP used in the Unix environment.

Further, the test control device 100 may be connected with other servers, such as the hospital information system 110 and the total laboratory automation system 120 through the network. Thus, the test control device 100 may be a computer system that receives a task performing request from another server and derives and provides a result of the task, or computer software (server program) installed for such a computer system.

The test control device 100 should be understood as a concept that encompasses a series of application programs operated on a server in addition to the above-described server programs and, in some case, various databases established inside or outside. Further, the test control device 100 may store and manage various types of information and data in a database. The database may be implemented inside or outside the test control device 100.

The network 130 is a network that connects the test control device 100 and the hospital information system 110 and/or total laboratory automation system 120 and may be a closed network, such as local area network (LAN) or wide area network (WAN) or an open network, such as the Internet. The Internet may mean a global open computer network structure that provides the TCP/IP protocol and several services present on the higher layer, i.e., hypertext transfer protocol (HTTP), Telnet, file transfer protocol (FTP), domain name system (DNS), simple mail transfer protocol (SMTP), simple network management protocol (SNMP), network file service (NFS), or network information service (NIS).

The test control device, method and recording medium briefly described above are described in more detail below.

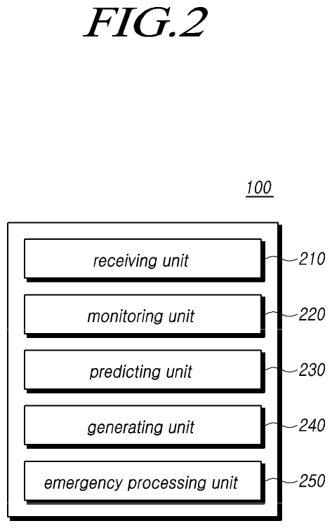

FIG. 2 is a view illustrating a configuration of a test control device according to an embodiment of the disclosure.

Referring to FIG. 2, according to an embodiment of the disclosure, a test control device 100 includes a receiving unit 210 receiving operation information from a hospital information system (HIS) and a total laboratory automation system (TLA), a monitoring unit 220 calculating per-progress step processing time information for each test performed in a laboratory based on the received operation information and monitoring a test progress process of the laboratory based on the processing time information, a predicting unit 230 predicting a test end time of each specimen from device state information and specimen sample information collected in real-time using a prediction model trained with operation information updated at predetermined periods, and a generating unit 240 generating output information for visualizing and displaying status information about the laboratory. The test control device 100 further includes an emergency processing unit 250 processing the sample of the specimen requested as emergency by an emergency process.

According to an embodiment, the receiving unit 210 may receive operation information from the hospital information system (HIS) and the total laboratory automation system (TLA). As an example, the receiving unit 210 may receive specimen sample information from the hospital information system and receive device state information from the total laboratory automation system. Accordingly, the operation information may include the specimen sample information and the device state information. For example, the operation information may be received at different periods depending on the times when detailed information included in the operation information is generated. The received operation information may be stored in a table structure which is divided per type of detailed information. This is described below in greater detail with reference to FIG. 2.

According to an embodiment, the monitoring unit 220 may calculate the processing time information per progress step for each test performed in the laboratory based on the operation information and monitor the test progress process of the laboratory based on the calculated processing time information. For example, the monitoring unit 220 may calculate the mean and standard deviation of the per-progress step processing times for each test during a predetermined period. The monitoring unit 220 may compare the retention time of the sample in progress in the real-time current step and the calculated mean and standard deviation, monitoring whether the test progress process corresponding to the current step has an abnormality. For example, the monitoring unit 220 may calculate and store the mean and standard deviation of the per-progress step processing times for each detailed test code included in the operation information for the past 30 days. However, the predetermined period is exemplified as 30 days, but is not limited thereto.

As another example, the monitoring unit 220 may determine whether the sample in progress in the current step falls outside the range corresponding to a specific magnification of the standard deviation with respect to the mean calculated from the specimen sample information under the same condition. When it is determined that the sample in progress falls outside the range, the monitoring unit 220 may detect an abnormality in the test progress process corresponding to the current step. For example, the monitoring unit 220 may calculate the mean and standard deviation of the per-progress step processing times from the specimen sample information about the specimens having the same condition regarding the specimen area, current specimen state, type, time, and holiday or not. If the retention time of the sample in progress exceeds the sum of the specific magnification and the mean and standard deviation of the per-progress step processing times, the monitoring unit 220 may detect an abnormality in the test progress process. The specific magnification may be arbitrarily set by the user. As a specific example, if it is determined that 2 standard deviation (2SD) is exceeded with respect to the mean of the per-progress step processing times for 30 days, the monitoring unit 220 may detect an abnormality in the test progress process.

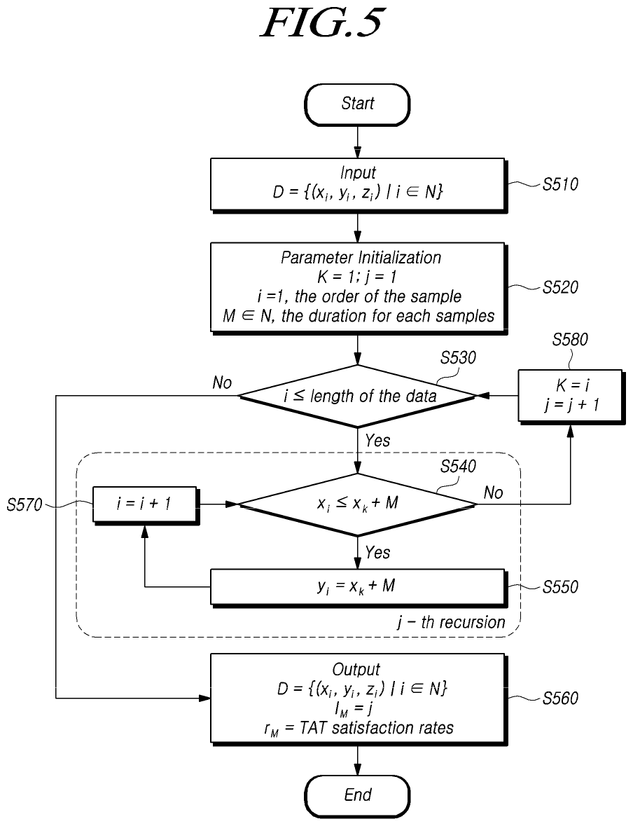

As another example, the monitoring unit 220 may monitor the test progress process of the emergency room sample based on the processing time (also referred to as turnaround time (TAT)) of the emergency room sample. For example, the monitoring unit 220 may determine the processing time of the emergency room sample based on the processing time information for the entire period and the limit time information per condition. The monitoring unit 220 may monitor the test progress process of the emergency room sample based on the result of determination. As a specific example, the monitoring unit 220 may calculate the per-condition limit time information having time, day, and real-time workload as conditions, based on the past information for a predetermined period. The limit time is the time when the provisional reception goes over to the reception and may mean the optimal time of being able to increase workload and reducing the task switching frequency while observing the processing time of the emergency room sample. Although emergency room is exemplified for description purposes, the processing time may also be applicable to other fields, such as in-hospital inpatient and outpatient care. The process of calculating the limit time information is described below in more detail with reference to FIG. 5.

According to an embodiment, the predicting unit 230 may predict the test end time of each specimen from the specimen sample information and device state information collected in real-time, using the prediction model trained through the operation information updated at predetermined periods. As an example, the predicting unit 230 may predict the test end time of each specimen through multivariate time series regression analysis using specific information included in the operation information as variables. Here, the prediction model may be a tree-based gradient boosting model. For example, the predicting unit 230 may use blood collection time, provisional reception time, reception time, test prescription, laboratory saturation at the time of blood collection, where the person whose blood is collected belongs, test device, test items, number of test items, day of the blood collection, or the presence or absence of a test error among the operation information. The predicting unit 230 may relearn and update the prediction model based on the operation information updated every 30 minutes and perform prediction using the latest updated prediction model. Accordingly, the predicting unit 230 may provide an effect of applying the prediction model most appropriate for the current situation. However, the predetermined period is exemplified as 30 minutes, but is not limited thereto.

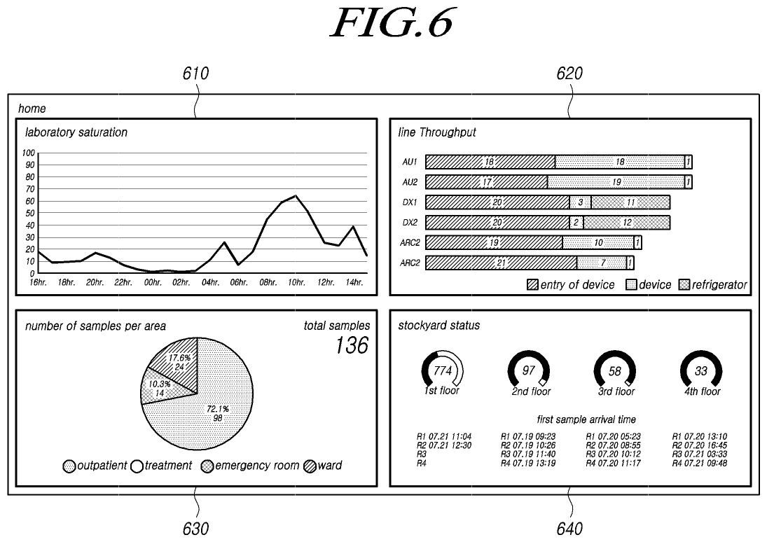

According to an embodiment, the generating unit 240 may generate output information for visualizing and displaying the status information about the laboratory. As an example, the generating unit 240 may generate output information regarding the laboratory status information including at least one piece of information among laboratory saturation information generated based on specimen quantity information requested from the laboratory, throughput information, number-of-samples-per-area information, stockyard status information, specimen lookup information, total laboratory automation (TLA) statistical information, and specimen status information. As another example, the generating unit 240 may further generate output information about detection information about whether there is an abnormality in the test progress process, prediction information for predicting the test end time, and management information about the emergency room sample. This is described below in greater detail with reference to FIGS. 6 to 9.

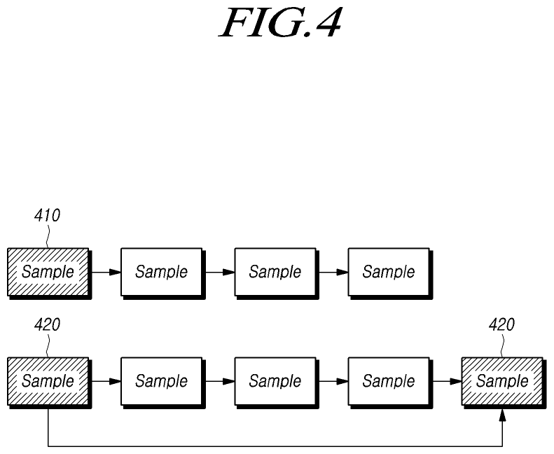

According to an embodiment, the emergency processing unit 250 may process the sample of the emergency-requested specimen via an emergency process. For example, the emergency processing unit 250 may detect the sample of the specimen emergency-requested by an outpatient nurse, transport it to a separate area, and process it first, i.e., via an emergency process. As another example, in a specific case, the emergency processing unit 250 may determine to process the sample of the non-emergency requested specimen via an emergency process. For example, the emergency processing unit 250 may compare the patient's treatment time with the test end time and, if it is determined that the test cannot be completed within the treatment time, the emergency processing unit 250 may determine to process the patient's specimen via an emergency process. This is described below in greater detail with reference to FIG. 4.

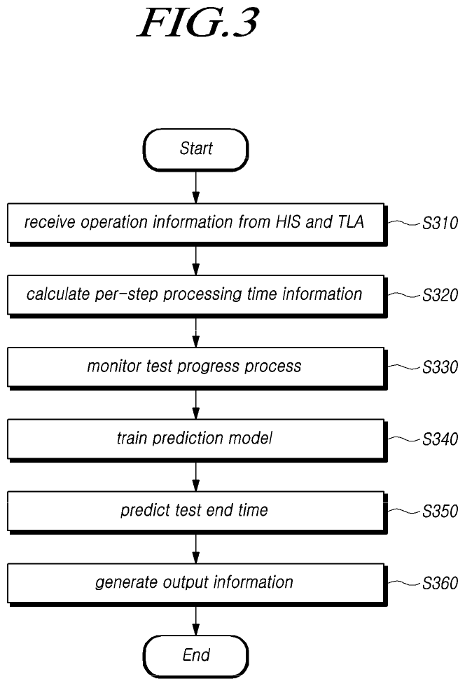

FIG. 3 is a flowchart illustrating an overall operation of a test control method according to an embodiment of the disclosure.

Referring to FIG. 3, according to an embodiment of the disclosure, the receiving unit 210 of the test control device may receive operation information from the hospital information system and the total laboratory automation system (S310). For example, the receiving unit 210 may receive operation information at different periods according to the generation times in conjunction with the hospital information system and the total laboratory automation system. Further, the receiver 210 may store the received operation information in a table structure divided by type. Therefore, in contrast to the conventional art in which basic management is possible only on the sample inserted to the total laboratory automation system, the embodiment of the disclosure interworks with the information system and total laboratory automation system which generates various pieces of information and may utilize them without device limitations in the laboratory. For example, the receiving unit 210 may receive XML format information at regular periods, parse information suitable for a table structure, and store it. The period when the receiver 210 receives the operation information may be expressed as shown in Table 1.

TABLE 1

| type | periods |
|---|---|
| data on the day | every 3 minutes |
| data before one day | on time each hour |
| data before 2-7 days | every 4 hours |
| data before 8-14 days | every 6 hours |
| data before 15-30 days | everyday |

The operation information received by the receiving unit 210 may be stored in a table structure as shown in Table 2. The detailed information included in the operation information may be generated from a source, such as Table 2.

TABLE 2

| table name | function | source |
|---|---|---|
| nursing_dept_emergency | emergency-requested | HIS |
| | patient registration table | |
| nursing_dept_monitoring | outpatient care | HIS |
| | patient-of-interest | |
| | registration table | |
| specimens | retain XML data | HIS |
| specimens_detail | retain XML detailed data | HIS |
| statistics* | store various statistical | HIS, TLA |
| | data | |
| system_settings* | store setting values | HAS |
| tat_statistics* | store TAT statistical data | calculate using |
| | | specimens_detail |
| tla_line_status | store TLA line state | TLA |
| users | user information | HAS |

As another example, the information included in Statistics and Tat_statistics which are table names may be information stored again by periodically performing calculations using data stored in the table structure. Specifically, Statistics may be information resultant from calculating, e.g., the number of samples processed by time in the laboratory. This may be utilized to predict the test saturation and test end time for the overall status of the laboratory. Tat_statistics may be information obtained by calculating the mean and standard deviation of per-step required times (collection-provisional reception, provisional reception-reception, reception-final reporting), with all detailed tests subdivided per specimen area (emergency room, outpatient care, ward), type, time, holiday or not, TAT observed or not, and specimen state (blood collection, provisional reception, reception) based on specimens detail. This may be utilized as essential information for the predicting unit 230. However, the table names are an example and are not limited thereto.

According to an embodiment, the monitoring unit 220 of the test control device may calculate processing time information for each processing step for each calculated test based on the operation information (S320). For example, the monitoring unit 220 may calculate the mean and standard deviation of the per-progress step processing times for 30 days for each detailed test code and store them in the table structure. The name of the stored table may be tat_statistics in Table 2. As another example, the monitoring unit 220 may calculate the mean and standard deviation from the specimen sample information having the same conditions for test area, current specimen state, type, time, and holiday or not, as the sample in progress in the current step.

As another example, the monitoring unit 220 may determine the processing time of the emergency room sample based on the processing time information for the entire period and the limit time information per condition. For example, the per-condition limit time information may mean the optimal time when provisional reception goes over to reception, as calculated based on past information for a predetermined period under the conditions of time, day, and real-time workload. Further, the processing time information for the entire period may mean the mean and standard deviation of the times taken from provisional reception to final reporting, for 30 days per detailed test code.

According to an embodiment, the monitoring unit 220 of the test control device may monitor the test progress process based on the calculated per-progress step processing time information (S330). As an example, the monitoring unit 220 may determine whether the test progress process in the laboratory has an abnormality using the mean and standard deviation of the per-progress step processing times. For example, the monitoring unit 220 may monitor samples which exceed the sum of specific magnifications of the mean and standard deviation. Such samples may be displayed, as abnormal samples, on the biochemistry dashboard screen and the immunology dashboard screen. Therefore, the monitoring unit 220 may real-time monitor the test progress process on several hundreds of samples generated in the hospital and immediately identify an issue with the process, preventing the patient's accident.

As another example, the monitoring unit 220 may monitor the test progress process of the emergency room sample based on the calculated processing time of the emergency room sample. For example, the monitoring unit 220 may monitor the limit time which is the optimal time when provisional reception goes over to reception and monitor the test progress process of the emergency room sample in the laboratory. Specifically, the monitoring unit 220 may monitor the test progress process using the sum of specific magnifications of the mean and standard deviation of the times taken from provisional reception to final reporting on the samples tested within the emergency room sample processing time and having the same conditions.

According to an embodiment, the predicting unit 230 of the test control device may train the prediction model through the operation information updated at predetermined periods (S340). As an example, the predicting unit 230 may utilize operation information stored in the table structure, as variables, for multivariate time series regression analysis. For example, the variables may be information stored in specimens detail in the table structure. Specifically, the variables may be ones regarding blood collection time, provisional reception time, reception time, test prescription, laboratory saturation at the time of blood collection, where the person whose blood is collected belongs, test device, test items, number of test items, day of the blood collection, or the presence or absence of a test error. As another example, the predicting unit 230 may use a tree-based gradient boosting model as a prediction model. For example, the prediction model may be a model that is updated after retrained according to the specimens detail information which is updated in the table structure every 30 minutes, rather than fixed after trained once. Accordingly, the predicting unit 230 may provide an accurate prediction result using the model most appropriate for the current situation by using the prediction model updated in real-time or daily according to information.

According to an embodiment, the predicting unit 230 of the test control device may predict the test end time of each specimen (S350). For example, the predicting unit 230 may predict the test end time of each specimen using the specimen sample information and device state information as input data of the prediction model. For example, the predicting unit 230 may predict the test end time of each specimen from the specimen sample information and device state information collected in real-time in each step. Specifically, the specimen sample information is information obtained by real-time checking whether there is a sample in conjunction with the hospital information system, receiving and storing it in the table structure and may include information about the sample barcode, sample ID, sample state, and processing time per sample state. The device state information is information received and stored in conjunction with the total laboratory automation system and may include information about the TLA error flag, device error flag, treatment division, test item, test device, and information about the person whose blood is collected. As another example, the predicting unit 230 may predict the test end time from each step corresponding to the blood collection step, which is the initial time when the test is conducted, the provisional reception step, or the reception step.

According to an embodiment, the generating unit 240 of the test control device may generate output information for visualizing and displaying the status information about the laboratory (S360). As an example, the generating unit 240 may generate output information about detection information about whether there is an abnormality in the test progress process, prediction information for predicting the test end time, and management information about the emergency room sample. For example, the generating unit 240 may generate output information to change the color of the sample box according to the result of TAT determination and apply the management information about the emergency room sample. Specifically, when the emergency room sample is in the provisional reception state in the laboratory, the generating unit 240 may use the mean time and standard deviation from the provisional reception to reception. Further, when the emergency room sample is in the reception state in the laboratory, the generating unit 240 may use the mean time and standard deviation from the provisional reception to final reporting. For example, if the emergency room sample exceeds the mean time of the samples observing the TAT in each state, the generating unit 240 may generate output information to change the color of the corresponding sample box to blue. This may mean that the corresponding emergency room sample is in a state of having a room until it observes the TAT. If the emergency room sample exceeds the sum of the mean time of the samples observing the TAT and the standard deviation (1SD) in each state, the generating unit 240 may generate output information to change the color of the corresponding sample box to yellow. This may mean that the corresponding emergency room sample should go to the next step to observe the TAT. If the emergency room sample exceeds the sum of the mean time of the samples observing the TAT and twice the standard deviation (2SD) in each state, the generating unit 240 may generate output information to change the color of the corresponding sample box to red. This may mean that the corresponding emergency room sample is in a state of having difficulty in observing the TAT and should be processed first. However, the magnification of the standard deviation to be added to the mean time is an example and, without limitations thereto, it may be changed according to the user's setting.

FIG. 4 is a view illustrating an example of an emergency process processing operation of a test control device according to an embodiment of the disclosure.

Referring to FIG. 4, an operation in which the emergency processing unit 250 of the test control device processes via an emergency process according to an embodiment of the disclosure is described. As an example, the emergency processing unit 250 may process the sample 410 of the emergency-requested specimen via an emergency process. For example, the emergency processing unit 250 may detect the sample of the specimen, emergency-requested by an outpatient nurse, automatically when entering the total laboratory automation system, transport it to a separate area, and process it first, i.e., via an emergency process. In this case, it may be visualized and known that the corresponding sample 410 has arrived at the laboratory and is in a separate area.

As another example, in a specific case, the emergency processing unit 250 may determine to process the sample 420 of the non-emergency requested specimen via an emergency process. For example, the emergency processing unit 250 may compare the patient's treatment time with the test end time and, if it is determined that the test cannot be completed within the treatment time, the emergency processing unit 250 may determine to process the patient's specimen sample via an emergency process. Specifically, in a case where the test end time is 1 PM, but the reserved treatment time is 12 AM, although the patient's specimen sample is the sample 420 of the specimen not emergency-requested, the emergency processing unit 250 may determine to automatically process it via the emergency process.

FIG. 5 is a flowchart illustrating an algorithm used to determine a time of processing an emergency room sample by a test control device according to an embodiment of the disclosure.

Referring to FIG. 5, according to an embodiment of the disclosure, an algorithm used to determine the processing time (also referred to as a turnaround time (TAT)) of the emergency room sample by the monitoring unit 220 of the test control device may be described.

As an example, the test control device may input original data to find limit time information for each condition (S510). For example, the test control device may input the time data of the ith sample, which is a 1×3 row vector, as original data. Here, xᵢ may mean the provisional reception time of the ith sample, yᵢ may mean the reception time of the ith sample, and zᵢ may mean the last reporting time of the ith sample.

As an example, the test control device may initialize variables (S520). For example, the test control device may set the limit time M corresponding to the time of going from provisional reception to reception to 1. The test control device may set the variable corresponding to the task switching frequency j to 1 and set i and k of the order of samples to 1 for initialization.

For example, the test control device may determine the length of the data (S530). For example, the test control device may determine whether the order of the sample is equal to or less than the length of the data.

For example, if it is determined that the order of the sample is less than or equal to the length of the data, the test control device may determine the provisional reception time of the ith sample (S540). For example, if the test control device determines that the order of the sample is less than or equal to the length of the data, the test control device may determine whether xᵢ which is the provisional reception time of the ith sample is not more than the sum of xₖ which is the provisional reception time of the kth sample and the limit time M. Accordingly, the test control device may collect samples until the time which is the provisional reception time of the first sample plus one minute, in the initialized state.

As an example, if xᵢ is determined to be not more than the sum of xₖ and the limit time M, the test control device may obtain the value of the reception time of the ith sample (S550). For example, if xᵢ is determined to be not more than the sum of xₖ and the limit time M, the test control device may obtain the sum of xₖ and the limit time M as yᵢ.

As an example, if obtaining the value of the reception time of the ith sample, the test control device may increase the order of the sample by one (S570). For example, the test control device may determine the provisional reception time of the i+1th sample by increasing the order of the sample by 1. Here, the i+1th sample may mean the sample next in order.

As an example, if xᵢ is determined to exceed the sum of xₖ and the limit time M, the test control device may increase the task switching frequency by one (S580). For example, if xᵢ is determined to exceed the sum of xₖ and the limit time M, the test control device may increase the task switching frequency j by one, set k to the sample order i, and re-determine the length of the data.

For example, if the test control device determines that the order of the sample exceeds the length of the data, the test control device may output the TAT observance rate (rₘ) and the task switching frequency (j=lₘ) (S560). For example, the test control device may calculate M from 1 minute to 60 minutes and calculate the TAT observance rate (rₘ) and the task switching frequency (j=lₘ) based on past data. The test control device may calculate the limit time to minimize the task switching frequency, as 30 minutes, based on the TAT observance rate. Accordingly, it may be identified that as the limit time increases, the task switching frequency decreases, but the TAT observance rate increases. The so-calculated limit time may be used as follows. For example, if there are one or more samples in the provisional reception state which are approaching the limit time, the test control device may flicker the edge of the display portion of the emergency room sample management information 720 in red, informing the user. However, the limit time may be calculated in real-time from per-past time range data identical in current time, real-time workload, and day, and is not limited to 30 minutes.

FIG. 6 is a view illustrating an example of a main screen for describing output information of a test control device according to an embodiment of the disclosure.

Referring to FIG. 6, a main screen displaying the output information of the test control device according to an embodiment of the disclosure may be described. As an example, the test control device may generate output information about the status information in the laboratory and display it on the main screen. For example, the test control device may generate output information about laboratory saturation information 610, throughput information 620, number-of-samples-per-area information 630, and stockyard status information 640 and display it on the main screen.

As a specific example, the laboratory saturation information 610 may be saturation information measured based on the specimen quantity requested from the laboratory. The laboratory saturation information 610 may be calculated as a value obtained by calculating the ratio of the current number of samples to the maximum daily number of samples in the laboratory over the past one year and display it in graph. In the graph, the x axis may be the last 24 hours, and the y axis may be the calculated value. As another example, the throughput information 620 may be processing time information taken to process samples by each device in the laboratory. The throughput information 620 may include the mean processing time from the insertion of the sample to arrival at the test device in conjunction with the total laboratory automation system. The throughput information 620 may include the mean processing time of retention of the sample in the test device or the mean processing time from the end of the test to arrival at the sample storage refrigerator. As another example, the number-of-samples-per-area information 630 may be information regarding the number of samples requested by the outpatient, treatment, emergency room, or ward. The stockyard status information 640 may be real-time status information about four specimen storage refrigerators storing test-finished samples.

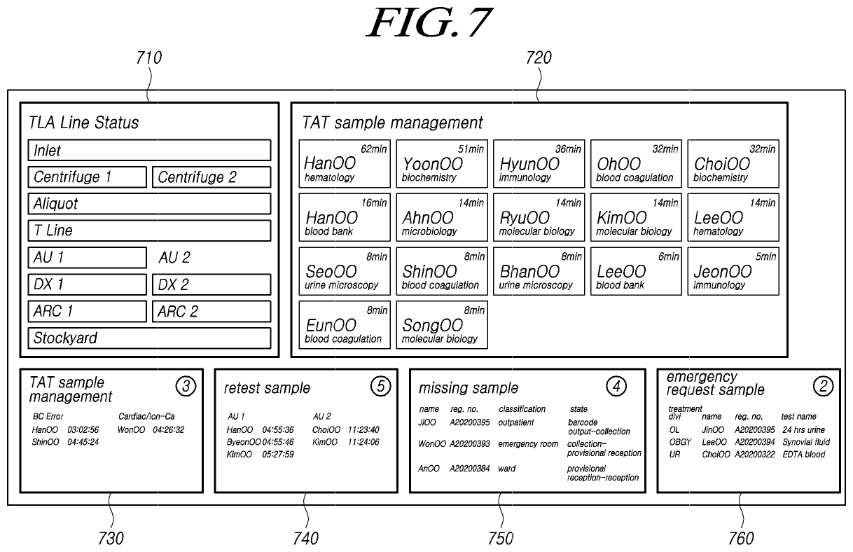

FIG. 7 is a view illustrating an example of a biochemical screen for describing output information of a test control device according to an embodiment of the disclosure.

Referring to FIG. 7, a biochemical screen displaying the output information of the test control device according to an embodiment of the disclosure may be described. As an example, the test control device may generate output information about a biochemical test and display it on the biochemical screen. For example, the test control device may generate output information for TLA status information 710 for the biochemical test, management information 720 about the emergency room sample, TLA error sample information 730, retest sample information 740, missing sample information 750, and emergency request sample information 760 and display it on the biochemical screen. The biochemical screen is characterized by having the largest number of specimens and in that results should be notified of within 1 to 2 hours.

As a specific example, the TLA status information 710 may be information about the real-time status of the device in the laboratory, which interworks with the total laboratory automation system. The TLA status information 710 may change the color according to the real-time status of the device to display priority. Green may indicate a normal operating state, gray may indicate a non-operational state, and red may indicate an error occurrence state. The TLA status information 710 may provide an effect of understanding the status of the entire line and equipment. As another example, the management information 720 about the emergency room sample may be information about the sample generated in the emergency room. The management information 720 about the emergency room sample may be displayed as a box for each sample. The box may contain information, such as the patient's name, the department that administers the test, and the elapsed time since provisional reception (TAT is calculated after provisional reception). The box may be displayed in three colors: blue, yellow, and red. The red box may be displayed first as it should be managed for TAT observance. As another example, the TLA error sample information 730 may be information about an error sample in which an exception occurs in the total laboratory automation system, rather than the device's own error. Further, the retest sample information 740 may be information about the sample for which a retest command is sent through the total laboratory automation system. The missing sample information 750 may be information about the sample whose state has not changed for a longer period of time than a reference time in the middle of the test from the total laboratory automation system and may include the patient's name, registration number, corresponding ward, and specimen state. The emergency request sample information 760 may be information about the emergency-requested sample and may be displayed in the order from the longest elapsed time after the emergency request. Further, if an actually emergency-requested sample is put into the device in the total laboratory automation system, it may be changed to red to indicate that the sample is taken out to a separate area.

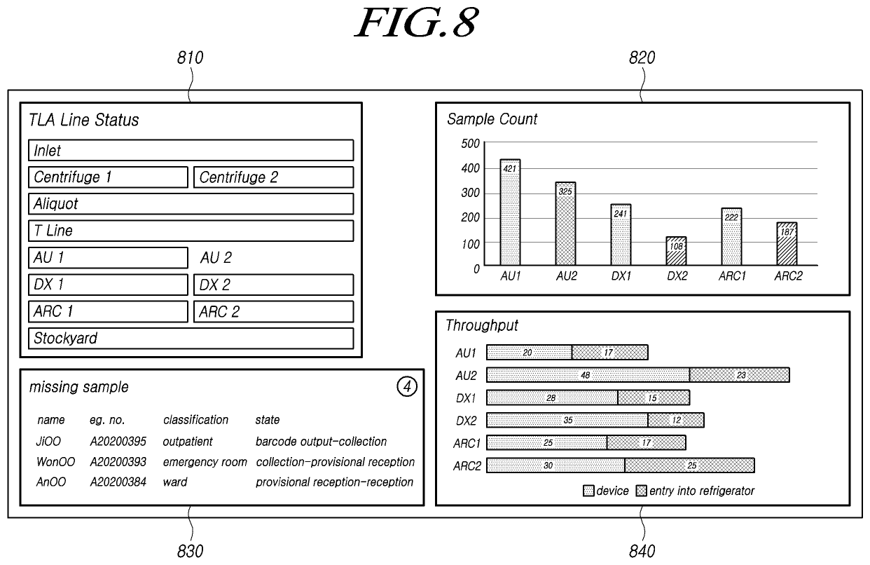

FIG. 8 is a view illustrating an example of an immunology screen for describing output information of a test control device according to an embodiment of the disclosure.

Referring to FIG. 8, an immunology screen displaying the output information of the test control device according to an embodiment of the disclosure may be described. As an example, the test control device may generate output information about an immunology test and display it on the immunology screen. For example, the test control device may generate output information for TLA status information 810 for the immunology test, number-of-samples information 820, missing sample information 830, and throughput information 840 and display it on the immunology screen. The immunology screen is characterized by having fewer specimens than the biochemical test.

As a specific example, the number-of-samples information 820 may be information regarding the number of samples that should currently be tested by the device in the laboratory interworking with the total laboratory automation system. The missing sample information 830 may be information about the sample whose state has not changed for a longer period of time than a reference time in the middle of the immunology test.

However, the screen is described as exemplifying the biochemical test and immunology test as the types of test, but is not limited thereto. Further, the information displayed on the screen is information selected by the user and may be set to be disposed in a changed position and size. Therefore, the screen is not limited to the above-described screen configuration.

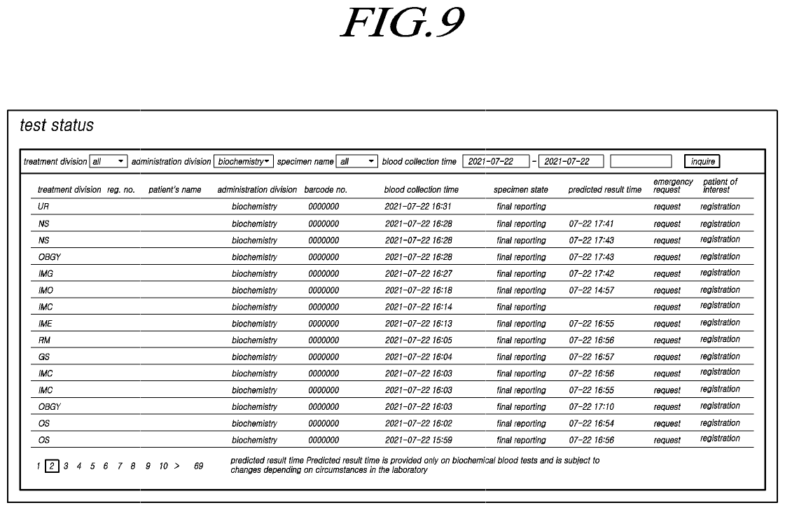

FIG. 9 is a view illustrating an example of a test status screen of predicting and using a test end time of a test control device according to an embodiment of the disclosure.

Referring to FIG. 9, a test status screen displaying output information generated by predicting the test end time of the test control device according to an embodiment of the disclosure may be described. As an example, the test control device may generate output information about the result of prediction of the test end time of each specimen and display it on the test status screen. For example, the test control device may generate output information about sample information about each specimen including predicted result time information, emergency request information, and information about the patient of interest and display it on the test status screen.

As a specific example, the predicted result time information may be information regarding the test end time of each specimen predicted by the predicting unit 230. The emergency request information may be information displaying the sample of the specimen emergency-requested through a collaboration system with an outpatient nurse. Further, the patient-of-interest information may be information identifying and displaying the patient selected to be notified in a pop-up when the test on the sample of the specimen is terminated.

A test control method that may be performed by the test control device described above in connection with FIGS. 1 to 9 is described below. No detailed description of some embodiments or some operations described in connection with FIGS. 1 to 9 may be given below for avoiding repetition of description, but the test control method may provide the above-described test control device in the same manner.

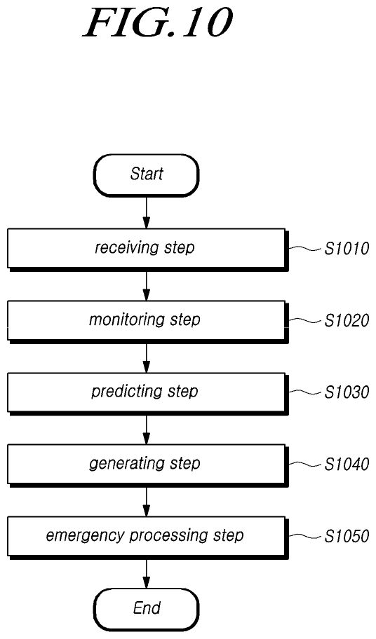

FIG. 10 is a flowchart illustrating a test control method according to an embodiment of the disclosure.

Referring to FIG. 10, the test control method of the disclosure may include a receiving step receiving operation information (S1010). According to an embodiment, the test control device may receive operation information from the hospital information system (HIS) and the total laboratory automation system (TLA). As an example, the test control device may receive specimen sample information from the hospital information system and receive device state information from the total laboratory automation system. Accordingly, the operation information may include the specimen sample information and the device state information. For example, the operation information may be received at different periods depending on the times when detailed information included in the operation information is generated. The received operation information may be stored in a table structure which is divided per type of detailed information.

The test control method of the disclosure may include a monitoring step monitoring the test progress process in the laboratory (S1020). According to an embodiment, the test control device may calculate the processing time information per progress step for each test performed in the laboratory based on the operation information and monitor the test progress process of the laboratory based on the calculated processing time information. For example, the test control device may calculate the mean and standard deviation of the per-progress step processing times for each test during a predetermined period. The test control device may compare the retention time of the sample in progress in the real-time current step and the calculated mean and standard deviation, monitoring whether the test progress process corresponding to the current step has an abnormality. For example, the test control device may calculate and store the mean and standard deviation of the per-progress step processing times for each detailed test code included in the operation information for the past 30 days. However, the predetermined period is exemplified as 30 days, but is not limited thereto.

As another example, the test control device may determine whether the sample in progress in the current step falls outside the range corresponding to a specific magnification of the standard deviation with respect to the mean calculated from the specimen sample information under the same condition. When it is determined that the sample in progress falls outside the range, the test control device may detect an abnormality in the test progress process corresponding to the current step. For example, the test control device may calculate the mean and standard deviation of the per-progress step processing times from the specimen sample information about the specimens having the same condition regarding the specimen area, current specimen state, type, time, and holiday or not. If the retention time of the sample in progress exceeds the sum of the specific magnification and the mean and standard deviation of the per-progress step processing times, the test control device may detect an abnormality in the test progress process. The specific magnification may be arbitrarily set by the user. As a specific example, if it is determined that 2 standard deviation (2SD) is exceeded with respect to the mean of the per-progress step processing times for 30 days, the test control device may detect an abnormality in the test progress process.

As another example, the test control device may monitor the test progress process of the emergency room sample based on the processing time (also referred to as turnaround time (TAT)) of the emergency room sample. For example, the test control device may determine the processing time of the emergency room sample based on the processing time information for the entire period and the limit time information per condition. The test control device may monitor the test progress process of the emergency room sample based on the result of determination. As a specific example, the test control device may calculate the per-condition limit time information having time, day, and real-time workload as conditions, based on the past information for a predetermined period. The limit time is the time when the provisional reception goes over to the reception and may mean the optimal time of being able to increase workload and reducing the task switching frequency while observing the processing time of the emergency room sample.

The test control method of the disclosure may include a predicting step predicting the test end time of each specimen (S1030). According to an embodiment, the test control device may predict the test end time of each specimen from the specimen sample information and device state information collected in real-time, using the prediction model trained through the operation information updated at predetermined periods. As an example, the test control device may predict the test end time of each specimen through multivariate time series regression analysis using specific information included in the operation information as variables. Here, the prediction model may be a tree-based gradient boosting model. For example, the test control device may use blood collection time, provisional reception time, reception time, test prescription, laboratory saturation at the time of blood collection, where the person whose blood is collected belongs, test device, test items, number of test items, day of the blood collection, or the presence or absence of a test error among the operation information. The test control device may relearn and update the prediction model based on the operation information updated every 30 minutes and use the latest updated prediction model. Accordingly, the test control device may provide an effect of applying the prediction model most appropriate for the current situation. However, the predetermined period is exemplified as 30 minutes, but is not limited thereto.

The test control method of the disclosure may include a generating step generating output information (S1040). According to an embodiment, the test control device may generate output information for visualizing and displaying the status information about the laboratory. As an example, the test control device may generate output information regarding the laboratory status information including at least one piece of information among laboratory saturation information generated based on specimen quantity information requested from the laboratory, throughput information, number-of-samples-per-area information, stockyard status information, specimen lookup information, total laboratory automation (TLA) statistical information, and specimen status information. As another example, the test control device may further generate output information about detection information about whether there is an abnormality in the test progress process, prediction information for predicting the test end time, and management information about the emergency room sample.

The test control method of the disclosure may include an emergency processing step processing via an emergency process (S1050). According to an embodiment, the test control device may process the sample of the emergency-requested specimen via an emergency process. For example, the test control device may detect the sample of the specimen emergency-requested by an outpatient nurse, transport it to a separate area, and process it first, i.e., via an emergency process. As another example, in a specific case, the test control device may determine to process the sample of the non-emergency requested specimen via an emergency process. For example, the test control device may compare the patient's treatment time with the test end time and, if it is determined that the test cannot be completed within the treatment time, the test control device may determine to process the patient's specimen via an emergency process.

Hereinafter, functions included in a recording medium storing a program for executing the test control method described above in connection with FIG. 10 are described. No detailed description of some embodiments or some operations described in connection with FIGS. 1 to 9 may be given below for avoiding repetition of description, but all of the functions corresponding to the above-described test control method may be executed.

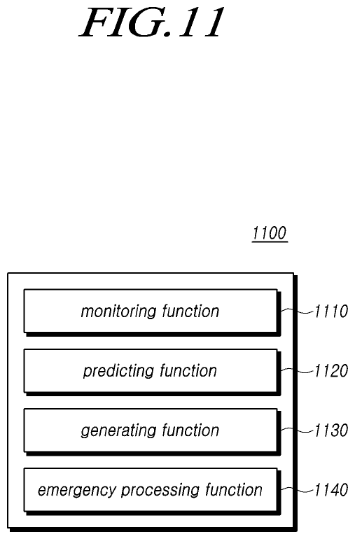

FIG. 11 is a view conceptually illustrating a configuration of a recording medium according to an embodiment of the disclosure.

According to an embodiment of the disclosure, a recording medium 1100 storing a program for executing a test control method may include a monitoring function 1110 calculating per-progress step processing time information for each test performed in a laboratory based on operation information received from a hospital information system (HIS) and a total laboratory automation system (TLA) and monitoring a test progress process of the laboratory based on the processing time information, a predicting function 1120 predicting a test end time of each specimen from device state information and specimen sample information collected in real-time using a prediction model trained with operation information updated at predetermined periods, and a generating function 1130 generating output information for visualizing and displaying status information about the laboratory. Further, the recording medium 1100 may further include an emergency processing function 1140 processing the sample of an emergency-requested specimen via an emergency process.

According to an embodiment, the monitoring function 1110 may calculate the processing time information per progress step for each test performed in the laboratory based on the operation information and monitor the test progress process of the laboratory based on the calculated processing time information. The operation information may be received from the hospital information system (HIS) and the total laboratory automation system (TLA). Further, the operation information may be received at different periods depending on the times when the detailed information included in the operation information is generated and be stored in a table structure divided per type of detailed information. For example, the monitoring function 1110 may calculate the mean and standard deviation of the per-progress step processing times for each test during a predetermined period and compare the retention time of the sample in progress in the current step and the calculated mean and standard deviation to monitor whether the test progress process has an abnormality. As a specific example, upon determining that the sample in progress in the current step falls outside a specific magnification of the standard deviation with respect to the mean calculated from the specimen sample information under the same condition, the monitoring function 1110 may detect an abnormality in the test progress process corresponding to the current step. The specimen sample information under the same condition may be sample information about the specimens having the same conditions regarding the specimen area, current specimen state, type, time, and holiday or no. The specific magnification of the standard deviation may be, but is not limited to, 2SD (standard deviation).

As another example, the monitoring function 1110 may determine the processing time of the emergency room sample based on the processing time information during the entire period and the per-condition limit time information and monitor the test progress process of the emergency room sample based on the result of determination. As a specific example, the test control device may calculate the per-condition limit time information having time, day, and real-time workload as conditions, based on the past information for a predetermined period. The limit time information is the time when the provisional reception goes over to the reception and may mean the optimal time of being able to increase workload and reducing the task switching frequency while observing the processing time of the emergency room sample.

According to an embodiment, the predicting function 1120 may predict the test end time of each specimen from the specimen sample information and device state information collected in real-time, using the prediction model trained through the operation information updated at predetermined periods. For example, the predicting function 1120 may predict the test end time of each specimen through multivariate time series regression analysis using specific information included in the operation information as variables. Here, the prediction model may be a tree-based gradient boosting model. As a specific example, the predicting function 1120 may use blood collection time, provisional reception time, reception time, test prescription, laboratory saturation at the time of blood collection, where the person whose blood is collected belongs, test device, test items, number of test items, day of the blood collection, or the presence or absence of a test error among the operation information. The predicting function 1120 may relearn and update the prediction model based on the operation information updated every 30 minutes and predict the test end time using the latest updated prediction model.

According to an embodiment, the generating function 1130 may generate output information for visualizing and displaying the status information about the laboratory. For example, the generating function 1130 may further generate output information about detection information about whether there is an abnormality in the test progress process, prediction information for predicting the test end time, and management information about the emergency room sample. As a specific example, the laboratory status information may include at least one piece of information among laboratory saturation information generated based on specimen quantity information requested from the laboratory, throughput information, number-of-samples-per-area information, stockyard status information, specimen lookup information, total laboratory automation (TLA) statistical information, and specimen status information.

According to an example, the emergency processing function 1140 may process the sample of the emergency-requested specimen via an emergency process. In a specific case, the emergency processing function 1140 may determine to process the sample of the non-emergency requested specimen via an emergency process. For example, the emergency processing function 1140 may detect the sample of the specimen emergency-requested by an outpatient nurse, transport it to a separate area, and process it first, i.e., via an emergency process. The emergency processing function 1140 may compare the patient's treatment time with the test end time and, if it is determined that the test cannot be completed within the treatment time, the emergency processing unit 250 may determine to process the patient's specimen via an emergency process.

The above-described test control method according to an embodiment of the disclosure may be implemented as an application (i.e., a program) installed by default on the test control device 100 or directly installed by the user and be recorded in a computer-readable recording medium, such as the test control device 100.

The program implementing the test control method according to an embodiment of the disclosure executes the monitoring function, the predicting function, the generating function, and the emergency processing function. The program may be recorded in the computer-readable recording medium and executed by the computer, executing the above-described functions.

As such, for the computer to read the program recorded on the recording medium and execute the test control method implemented as a program, the above-described program may include code coded in a computer language, such as C, C++, JAVA, or machine language, which the processor (CPU) of the computer may read.

Such code may include a function code related to a function defining the above-described functions or may include an execution procedure-related control code necessary for the processor of the computer to execute the above-described functions according to a predetermined procedure.

Further, the code may further include additional information necessary for the processor of the computer to execute the above-described functions or memory reference-related code as to the position (or address) in the internal or external memory of the computer the media should reference.

Further, when the processor of the computer needs to communicate with, e.g., another computer or a server at a remote site to execute the above-described functions, the code may further include communication-related code as to how the processor of the computer should communicate with the remote computer or server using the communication module (e.g., wired and/or wireless communication module) of the computer and what information or media should be transmitted/received upon communication.

The functional programs for implementing the disclosure and code and code segments related thereto may easily be inferred or changed by programmers of the technical field to which the disclosure pertains, considering, e.g., the system environments of the computer reading and executing the program.

Further, the computer-readable recording medium storing the above-described program may be distributed to computer systems connected via a network, and computer-readable codes may be stored and executed in a distributed manner. In this case, any one or more computers among the multiple distributed computers may execute some of the above-suggested functions and transmit the result of execution to one or more of other distributed computers, and the computer receiving the result may execute some of the above-suggested functions and provide the result of execution to other distributed computers.

The above-described computer-readable recording medium for executing the test control method according to an embodiment of the disclosure may include, e.g., a ROM, a RAM, a CD-ROM, a magnetic tape, a floppy disk, and an optical media storage device.

Further, the computer-readable recording medium storing an application, which is a program for executing the test control method according to an embodiment of the disclosure, may be a storage medium (e.g., hard disk) included in an application provider server including, e.g., an application store server and a web server related to the application or service, or the application provider server itself, or another computer storing the program or its storage medium.

The computer that may read the recording medium storing the application, which is a program for executing the test control method, according to an embodiment of the disclosure should be interpreted not only as a general PC, such as a general desktop or laptop computer, but also as a smart phone, a tablet PC, a personal digital assistant (PDA) and a mobile terminal, e.g., a mobile communication terminal, or any other devices capable of computing.

When the computer that may read the recording medium storing the application which is a program for executing the test control method according to an embodiment of the disclosure is a smartphone, a tablet PC, a personal digital assistant (PDA), a mobile communication terminal, or such a mobile terminal, the mobile terminal may download and install the application from an application provider server including an application store server or a web server and, in some cases, the application may be downloaded from the application provider server to a general PC and be then installed on the mobile terminal through a synchronization program.

Although it is described above that all of the components are combined into one or are operated in combination, embodiments of the disclosure are not limited thereto. One or more of the components may be selectively combined and operated as long as it falls within the scope of the aspects of the disclosure. Further, although all of the components may be implemented in their respective independent hardware components, all or some of the components may be selectively combined to be implemented in a computer program with program modules performing all or some of the functions combined in one or more hardware components. The codes and code segments constituting the computer program may be easily inferred by one of ordinary skill in the art to which the disclosure pertains. The computer program may be stored in computer readable media and be read and executed by a computer to implement embodiments of the disclosure. The storage medium of the computer program may include a magnetic recording medium, an optical recording medium, and the like.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the disclosure. Accordingly, the embodiments set forth herein are provided for illustrative purposes, but not to limit the scope of the disclosure, and should be appreciated that the scope of the disclosure is not limited by the embodiments. The scope of the disclosure should be construed by the following claims, and all technical spirits within equivalents thereof should be interpreted to belong to the scope of the disclosure.

What is claimed is:

1. A test control device, comprising:
a receiving unit receiving operation information from a hospital information system (HIS) and a total laboratory automation system (TLA);
a monitoring unit calculating per-progress step processing time information for each test performed in a laboratory based on the received operation information and monitoring a test progress process of the laboratory based on the processing time information;
a predicting unit predicting a test end time of each specimen from device state information and specimen sample information collected in real-time using a prediction model trained with the operation information updated at a predetermined period; and
a generating unit generating output information for visualizing and displaying status information about the laboratory.

2. The test control device of claim 1, further comprising an emergency processing unit processing a sample of an emergency-requested specimen via an emergency process,
wherein the emergency processing unit compares a patient's treatment time with the test end time and, upon determining that the test cannot be completed within the test time, determines to process the patient's specimen via the emergency process.

3. The test control device of claim 1, wherein the operation information is received at different periods depending on times when detailed information included in the operation information is generated and is stored in a table structure divided per type of the detailed information.

4. The test control device of claim 1, wherein the monitoring unit calculates a mean and standard deviation of per-progress step processing times for each test during a predetermined period, compares a retention time of a sample in progress in a current step with the mean and the standard deviation, and monitors whether the test progress process corresponding to the current step has an abnormality.

5. The test control device of claim 4, wherein upon determining that the sample in progress in the current step falls outside a specific magnification of the standard deviation with respect to the mean calculated from specimen sample information under a same condition, the monitoring unit detects the abnormality in the test progress process corresponding to the current step.

6. The test control device of claim 1, wherein the monitoring unit determines a processing time (turnaround time (TAT)) of an emergency room sample based on processing time information during an entire period and per-condition limit time information and monitors the test progress process of the emergency room sample based on a result of the determination.

7. The test control device of claim 1, wherein the predicting unit predicts the test end time of each specimen through multivariate time series regression analysis using specific information included in the operation information as a variable.

8. The test control device of claim 1, wherein the prediction model is a tree-based gradient boosting model.

9. The test control device of claim 1, wherein the laboratory status information includes at least one piece of information among laboratory saturation information generated based on specimen quantity information requested from the laboratory, throughput information, number-of-samples-per-area information, stockyard status information, specimen lookup information, total laboratory automation (TLA) statistical information, and specimen status information.

10. The test control device of claim 1, wherein the generating unit further generates the output information about detection information about the abnormality in the test progress process, prediction information for predicting the test end time, and management information about the emergency room sample.

11. A test control method, comprising:
a receiving step receiving operation information from a hospital information system (HIS) and a total laboratory automation system (TLA);
a monitoring step calculating per-progress step processing time information for each test performed in a laboratory based on the operation information and monitoring a test progress process of the laboratory based on the calculated processing time information;
a predicting step predicting a test end time of each specimen from specimen sample information and device state information collected in real-time, using a prediction model trained with the operation information updated at a predetermined period; and
a generating step generating output information for visualizing and displaying status information about the laboratory.

12. The test control method of claim 11, further comprising an emergency processing step processing a sample of an emergency-requested specimen via an emergency process,
wherein the emergency processing step compares a patient's treatment time with the test end time and, upon determining that the test cannot be completed within the test time, determines to process the patient's specimen via the emergency process.

13. The test control method of claim 11, wherein the operation information is received at different periods depending on times when detailed information included in the operation information is generated and is stored in a table structure divided per type of the detailed information.

14. The test control method of claim 11, wherein the monitoring step calculates a mean and standard deviation of per-progress step processing times for each test during a predetermined period, compares a retention time of a sample in progress in a current step with the mean and the standard deviation, and monitors whether the test progress process corresponding to the current step has an abnormality.

15. The test control method of claim 14, wherein upon determining that the sample in progress in the current step falls outside a specific magnification of the standard deviation with respect to the mean calculated from specimen sample information under a same condition, the monitoring step detects the abnormality in the test progress process corresponding to the current step.

16. The test control method of claim 11, wherein the monitoring step determines a processing time (turnaround time (TAT)) of an emergency room sample based on processing time information during an entire period and per-condition limit time information and monitors the test progress process of the emergency room sample based on a result of the determination.

17. The test control method of claim 11, wherein the predicting step predicts the test end time of each specimen through multivariate time series regression analysis using specific information included in the operation information as a variable.

18. The test control method of claim 11, wherein the prediction model is a tree-based gradient boosting model.

19. The test control method of claim 11, wherein the laboratory status information includes at least one piece of information among laboratory saturation information generated based on specimen quantity information requested from the laboratory, throughput information, number-of-samples-per-area information, stockyard status information, specimen lookup information, total laboratory automation (TLA) statistical information, and specimen status information.

20. The test control method of claim 11, wherein the generating step further generates the output information about detection information about the abnormality in the test progress process, prediction information for predicting the test end time, and management information about the emergency room sample.

21. A recording medium storing a program for executing a test control method, comprising:
a monitoring function calculating per-progress step processing time information for each test performed in a laboratory based on operation information received from a hospital information system (HIS) and a total laboratory automation system (TLA) and monitoring a test progress process of the laboratory based on the processing time information;
a predicting function predicting a test end time of each specimen from specimen sample information and device state information collected in real-time, using a prediction model trained with the operation information updated at a predetermined period; and
a generating function generating output information for visualizing and displaying status information about the laboratory.
